(19) United States
(12) Patent Application Publication
(10) Pub. No.: US 2007/0021640 A1
(43) Pub. Date: Jan. 25, 2007

(54) Method and apparatus for application of light to gums

(76) Inventor: Thomas Perez, Chicago, IL (US)

Correspondence Address: Orum & Roth LLC;Ste 1616, 53 W Jackson Blvd, Chicago, IL, 60604, US

(21) Appl. No.: 11/485,175

(22) Filed: Jul. 12, 2006

Related U.S. Application Data
(63) Continuation-in-part of application No. 11/248,995, filed on Oct. 12, 2005.
Application No. 11/248,995 is a continuation-in-part of application No. 11/235,652, filed on Sep. 26, 2005.
Application No. 11/235,652 is a continuation-in-part of application No. 11/140,272, filed on May 27, 2005.
Application No. 11/140,272 is a continuation-in-part of application No. 11/076,169, filed on Mar. 9, 2005.
Application No. 11/140,272 is a continuation-in-part of application No. 10/926,209, filed on Aug. 25, 2004.
(60) Provisional application No. 60/503,678, filed on Sep. 17, 2003.

(51) Int. Cl. A61N 5/00

(52) U.S. Cl. 600/1

(57) ABSTRACT

Light having one or more therapeutic wavelengths is applied to a patient's gums. A mouthguard directing the UV-A, UV-C or both UV-A and UV-C light to the patient's gums is placed in the patient's mouth. The mouth guard is placed over the teeth and gums or between the gums and lips. The gums are then illuminated.

This application is a continuation in part of U.S. application Ser. No. 11/248,995 foiled 12 Oct. 2005 that is a continuation in part of U.S. application Ser. No. 11/235,652 filed 26 Sep. 2005 that is a is a continuation in part of U.S. application Ser. No. 11/140,272 filed 27 May 2005 that is a continuation in part of U.S. application Ser. No. 11/076,169 filed 9 Mar. 2005 and U.S. application Ser. No. 10/926,209 filed 25 Aug. 2004 that claimed the benefit of U.S. Provisional Application No. 60/503,678 filed Sep. 17, 2003.

### BACKGROUND OF THE INVENTION

1. Field of the Invention

The present invention relates to a method and apparatus for providing light to gums. More specifically, the present invention relates to a method and apparatus for irradiation of the gums.

2. Background of the Prior Art

Ultraviolet (UV) light can be used to treat a multitude of medical problems, including for example bacterial, viral and fungal infections, poisoning, fatigue, Alzheimer's disease, allergies and asthma, rheumatic diseases and arthritis, diabetes, hepatitis, and cancer. UV light sterilizes the blood and acts as an antibiotic.

The UV light is applied either to the patient's skin or directly to the blood. If the UV light is applied to the skin it is typically provided to the patient's skin either with a wrap or lamp.

UV light is commonly used to treat jaundiced babies. Because infant's skin is thin and the blood vessels are close to the surface, UV light is typical applied to the skin when treating jaundiced babies.

Applying the UV light directly to a patient's blood supply is known as photoluminescence or UV blood illumination (UBI). UV blood illumination increases oxygen, destroys toxins and boosts the immune system.

In prior art UBI, a small amount of blood is drawn from the patient, up to about 250 cc. The body has about 5.6 L of blood. The blood that is drawn travels through a cuvette or glass chamber. The blood is repeatedly illuminated with UV light and then returned to the body. The process is repeated, typically a day or several days later. These treatments are time consuming, and require regular trips to a medical facility. In addition, trained personal must be available to provide the treatments.

There is a need for a method of providing UV light to specific target areas of the body. There is a need for a system that is convenient for the patient, which does not require regular doctor visits. There is a need for a simple system that can be used by the patient in his home.

There is a need for a system that allows for regular treatments such as pulsed treatment or automatic periodic treatments.

### SUMMARY OF THE INVENTION

The present invention is a mouthguard that irradiates the gums. The mouthguard has a battery or other power supply and a light source. Alternatively, the mouthguard can be connected to a power supply and/or to a light source. The light source emits light at one or more therapeutic wavelengths. Preferably, the light is UV light at one or more therapeutic wavelengths. The UV light source is typically LEDs that emit UV-A or UV-C light or a combination of UV-A and UV-C light. The light pack or device is inserted into the patient's mouth, preferably over the gums. In one embodiment, the mouthguard preferably has one or more medical grade UV light bulbs. In one embodiment, the bulb is a cold cathode fluorescent bulb. In another embodiment, fiber optic strand(s) run through a tube to illuminate the mucous membrane under the tongue 174. Capillaries are plentiful and close to the surface in the gums, thus illuminating in addition to illuminating the gums directly, the patient's blood will also be illuminated.

### BRIEF DESCRIPTION OF THE DRAWINGS

FIG. 1 is a light irradiation delivery device.

FIG. 2 is the light irradiation delivery device of claim 1 inserted into a patient's mouth.

FIG. 3 is an alternative embodiment of the light irradiation delivery device.

FIG. 4 is an alternative embodiment of the light irradiation delivery device.

FIGS. 5a and 5b are alternative embodiments of the light irradiation delivery device.

FIGS. 6a-6c are an alternative cold cathode fluorescent bulb for use with the sublingual irradiation delivery devices.

FIG. 7 is a top view of an alternative embodiment of the inventive mouthguard.

FIG. 8 is a front view of the mouthguard of FIG. 7.

FIG. 9 is a side view of the mouthguard of FIGS. 7.

FIG. 10 is a front view of an alternative embodiment of the mouthguard.

FIG. 11 is a back view of the mouthguard of FIG. 10.

FIG. 12 is a top view of an alternative embodiment of the mouthguard.

FIG. 13 is a side view of the mouthguard of FIG. 12.

FIG. 14 is a bottom view of an alternative embodiment of the mouthguard.

FIG. 15 is a top view of the mouthguard of FIG. 14.

FIG. 16 is a side view of the mouthguard of FIG. 14.

FIG. 17 is a cross section of a patient wearing the mouthguard of FIGS. 14-16.

FIG. 18 is a cross section of a patient wearing the mouthguard of FIGS. 10-11.

FIG. 19 is a cross section of patient wearing an alternative embodiment of the mouthguard of FIGS. 12-13.

FIG. 20 is a cross section of a patient wearing the mouthguard of FIGS. 12-13.

FIG. 21 is a cross section of a patient wearing an alternative embodiemtn of the mouthguard of FIGS. 14-16.

FIG. 22 is a side view of an alternative embodiment of the mouthguard of FIG. 14.

FIG. 23 is a side view of an alternative embodiment of the mouthguard of FIG. 14.

### DETAILED DESCRIPTION OF THE INVENTION

Light at one or more therapeutic wavelength, such as ultraviolet light (UV), is used to treat many diseases including infections, poisoning, fatigue, allergies, hepatitis, cancer and HIV. UV light increases the oxygen combining power of the blood, destroys toxins, viruses, fungi, bacteria, and boosts the immune system. UV light also sterilizes the blood and acts as an antibiotic. Capillary exposure of the mucus membranes is significantly greater than other exposed body surfaces. Greater capillary exposure allows for greater penetration of the ultraviolet spectrum. The mouth and gums are made of mucus membrane making them useful surfaces to be treated with therapeutic light irradiation.

There is a correlation between gum disease and heart disease and stroke. People with gum disease are twice as likely to have heart problems as those without gum disease. Further, gum disease may exacerbate an existing heart condition. Researchers believe that oral bacteria may enter the blood stream and contribute to clot formation by combining with fatty plaque. Other researchers believe that the inflammation of the gums increases fatty plaque build-up.

The present invention is a device and method to irradiate gum tissue. Preferably, UV light at one or more therapeutic wavelength is utilized. More preferably, the light is either UV-A or UV-C or a combination of UV-A and UV-C light. For some conditions and/or diseases UV-A light is more effective than UV-C and for other conditions and/or diseases UV-C light is more effective than UV-A light. The wavelengths or wavelengths of light to be used to treat the patient maybe selected based on the wavelength or wavelength that will best treat the condition(s) or disease(s) of the patient.

Mouthguard 40 mouthguard 142 is placed into the patient's 10 mouth for treatment. Mouthguard 40 may be placed over the gums, over gums and teeth 102, between the tongue 100 and the gums, or fit in the pallet and the back of the gums. The gum tissue is then irradiated using one or more therapeutic wavelengths of light.

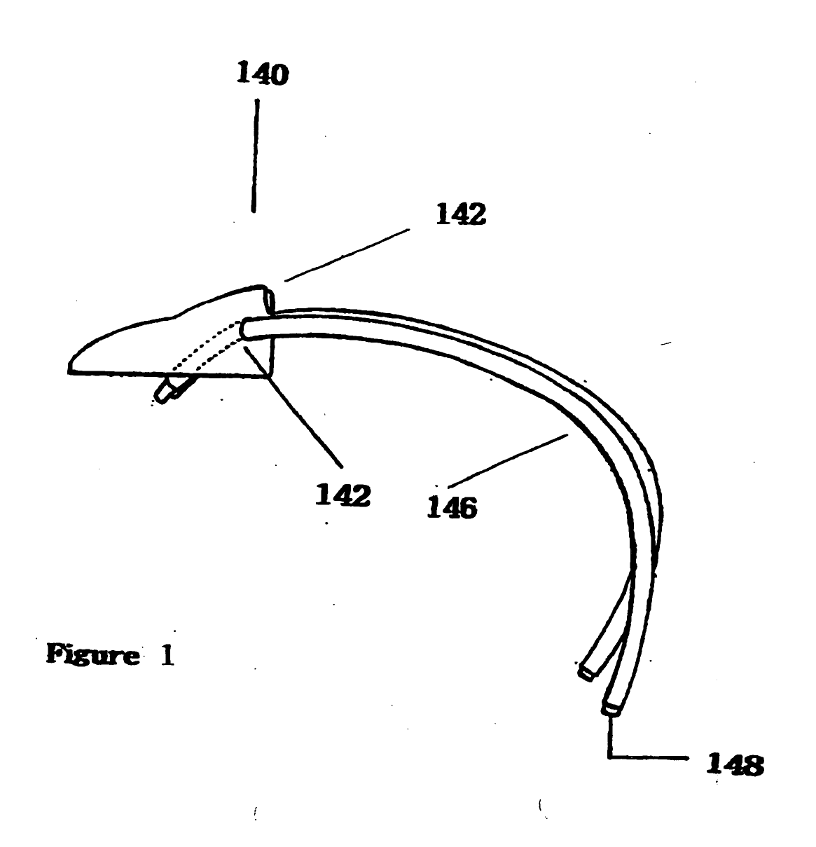

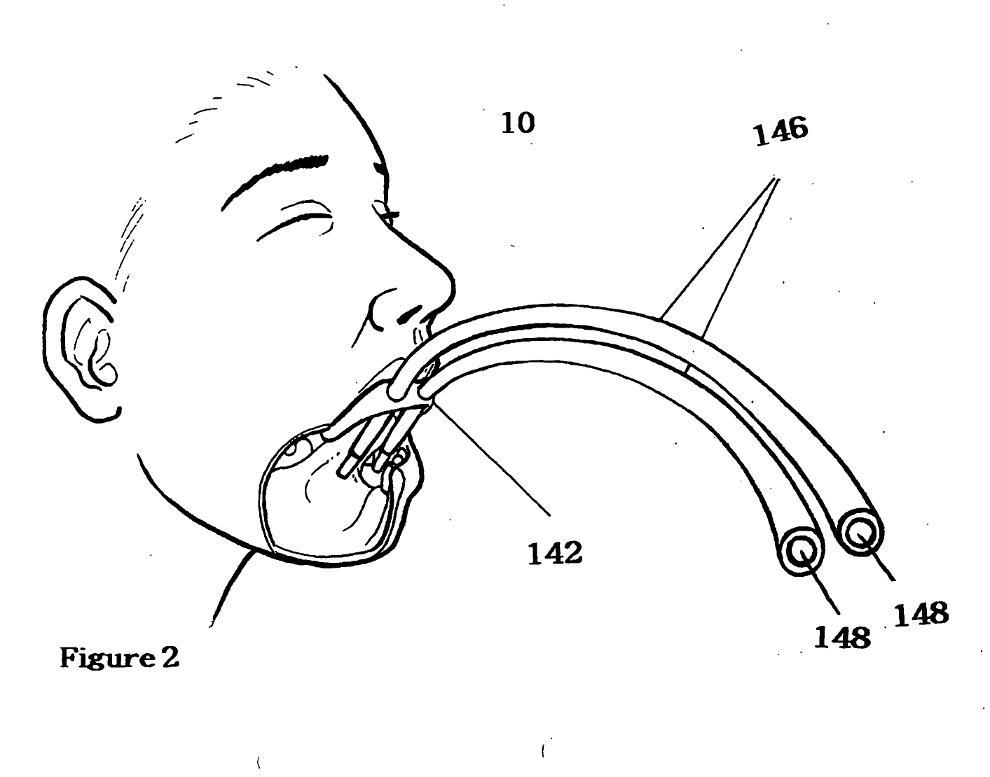

In one embodiment of the invention, as shown in FIGS. 1 and 2, light at one or more therapeutic wavelengths is administered under the tongue 174, on the gums, or on the pallet. The capillaries on interior of the mouth are close to the surface. These capillaries are very sensitive. The light device 140 comprises a mouthguard 142 for holding and aligning the light source under the tongue 174. Mouthguard 142 is inserted into patient's 10 mouth. The mouthguard 142 has at least one aperture 144 through which tubing 146 is inserted. Tubing 146 is preferably plastic tubing and is preferably flexible. The tubing may be adjustably inserted through the at least one aperture 144 to allow for individual adjustment by the patient 10 or doctor to a preferred treatment location such as between the tongue 100 and the gums, under the patient's tongue 174, or directed toward a specific area of the gums or mouth. Alternatively, tube 146 can be mounted in the preferred position such that each time the mouthguard 142 is used, the light is administered at the same location. Preferably, mouthguard 142 is molded to the shape of patient's mouth. There are preferably fused silica fiber optic bundles 148 in the tubing 144. Fused silica fiber optic bundles 148 are preferred because they do not emit any heat. Optionally, the light source is a cold cathode fluorescence bulb. One or more low voltage cables are used to power the cold cathode fluorescent bulb. The fiber optic bundles 148 preferably deliver UV light at a therapeutic wavelength sublingually, to the gums and/or to the pallet. The light is preferably UV-A, UV-C or a combination thereof. In yet another alternative, light source is a LED or small light bulb at the end of the flexible tube adapted to directly irradiate the mucus membrane under the tongue 174. This delivery system is preferred for relatively young patients.

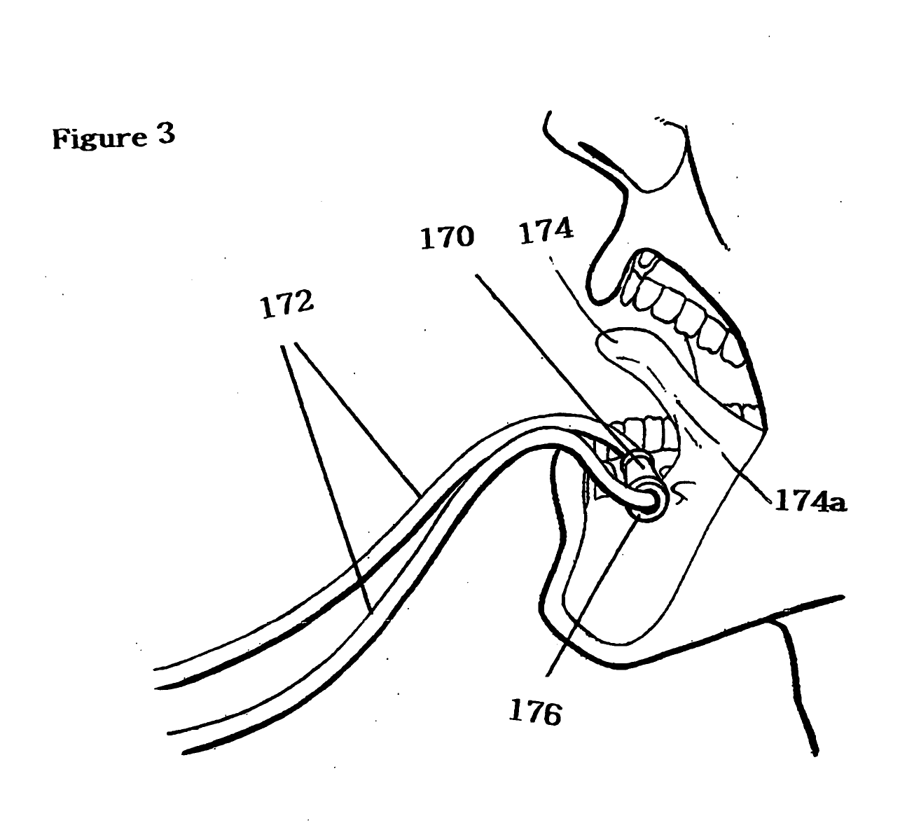

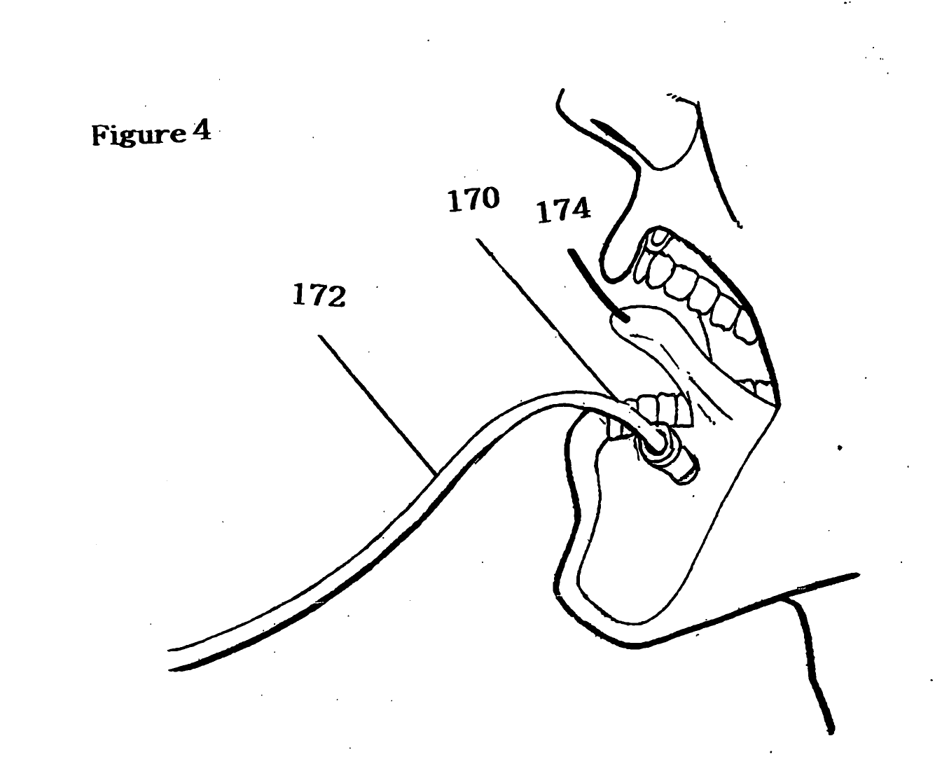

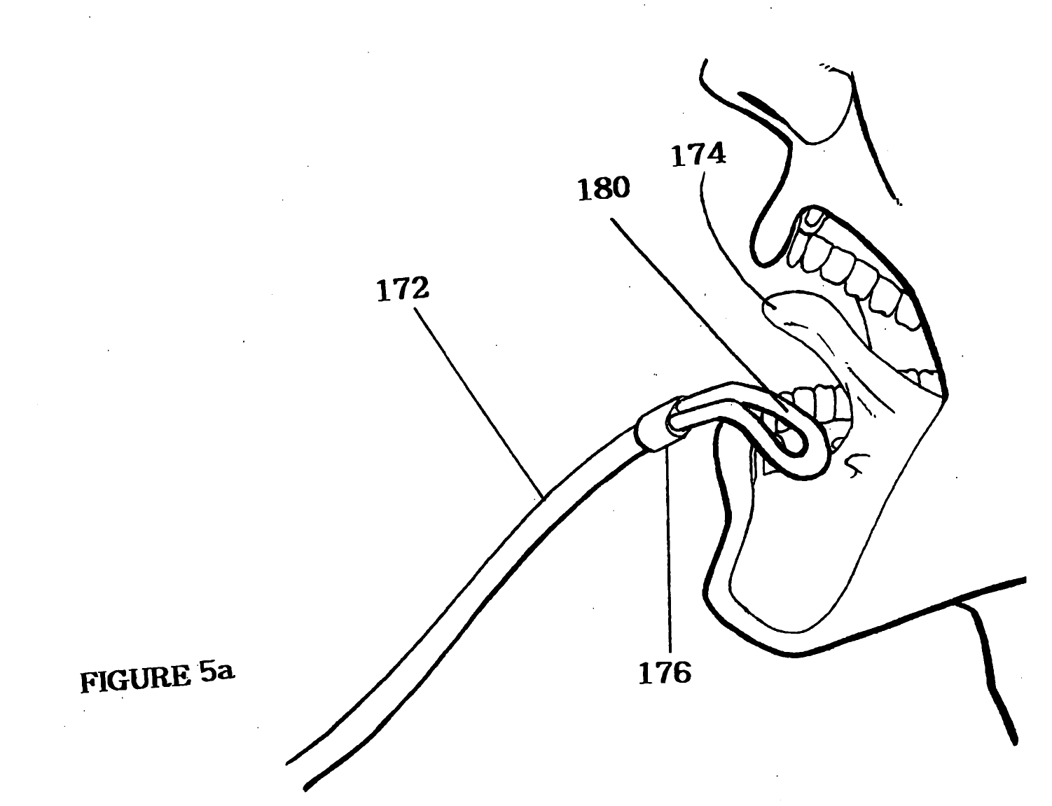

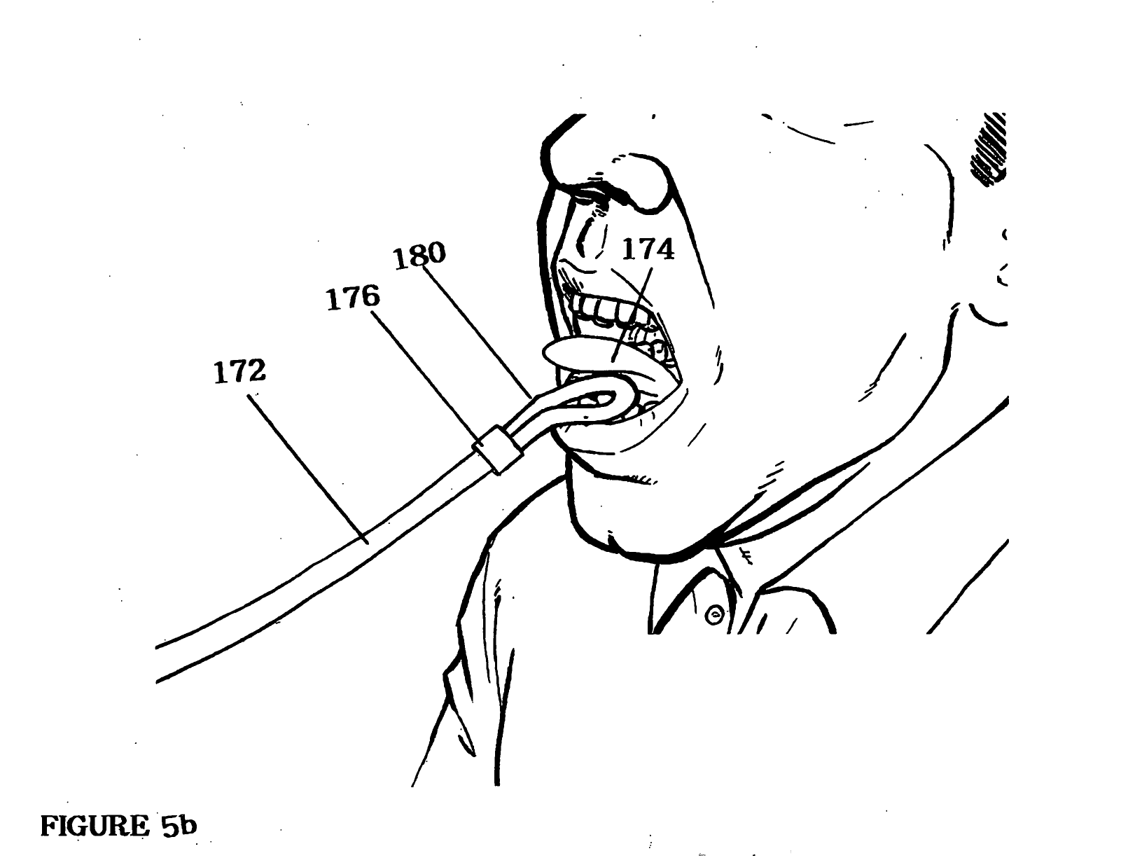

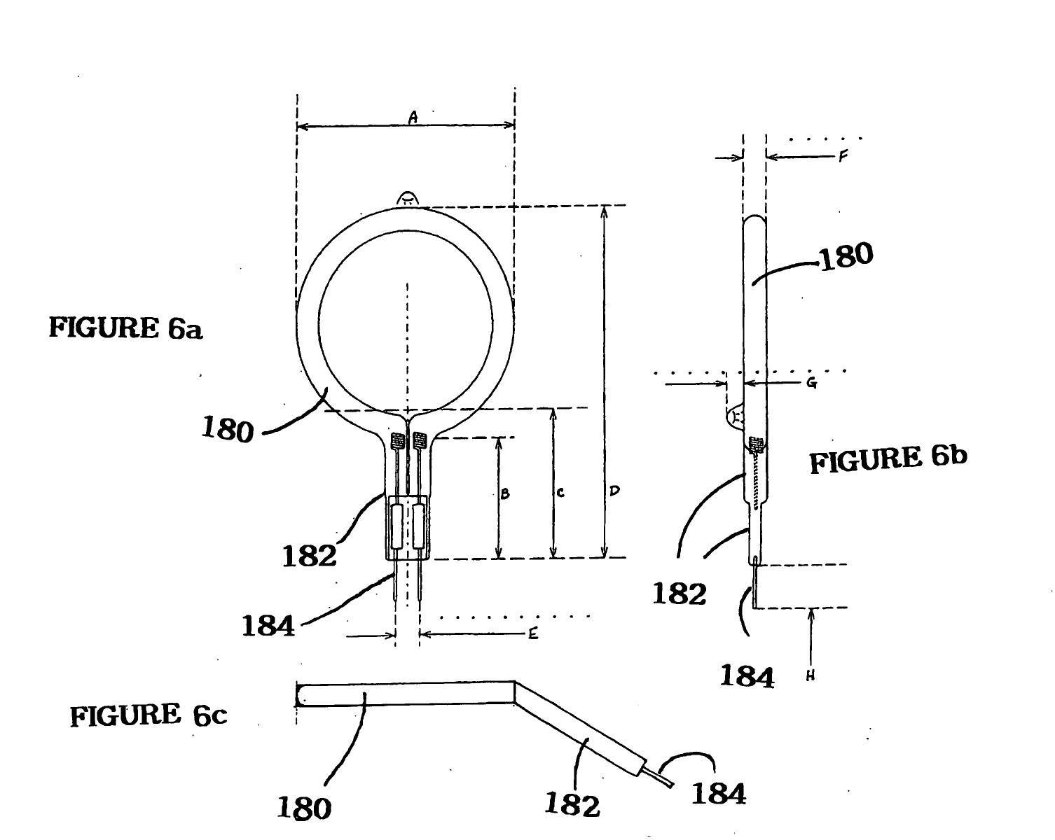

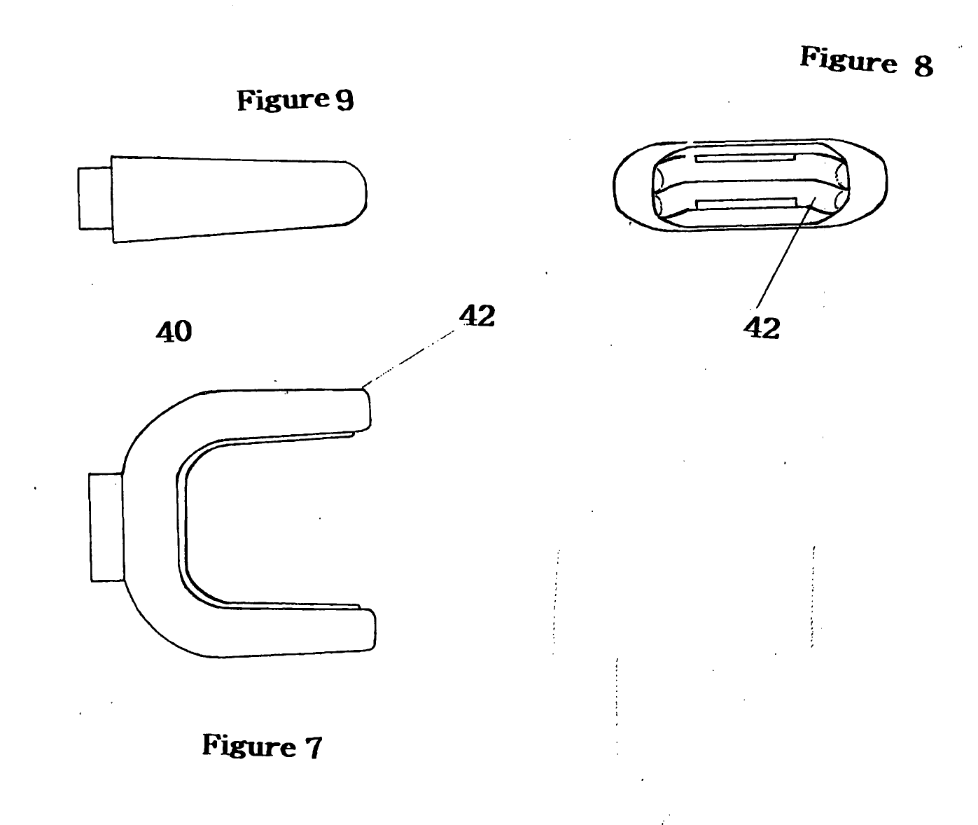

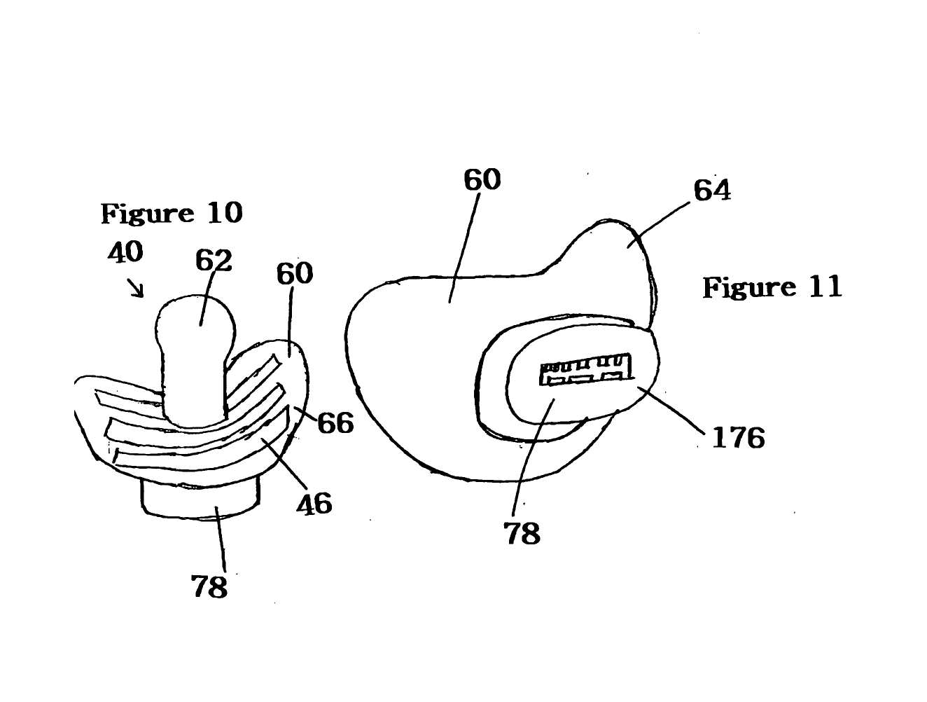

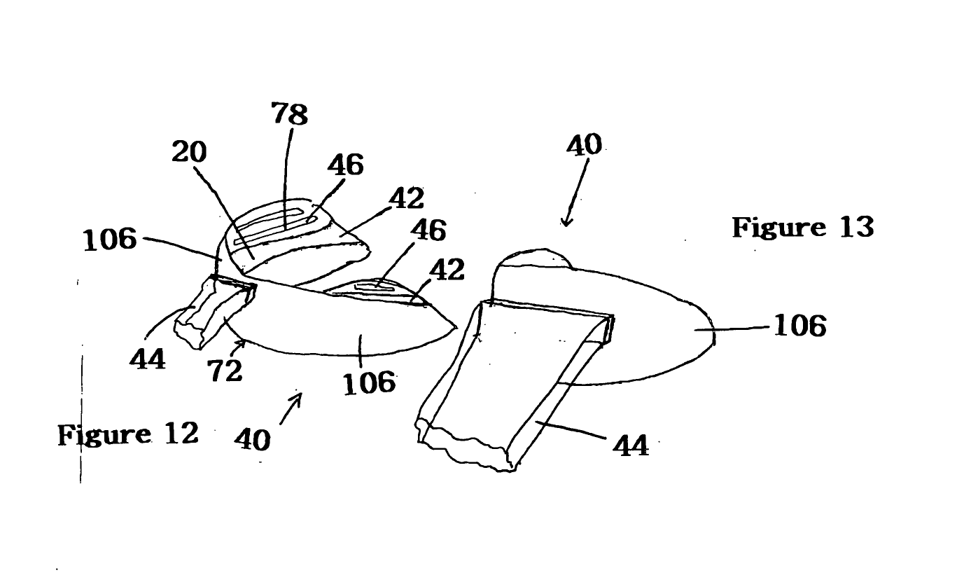

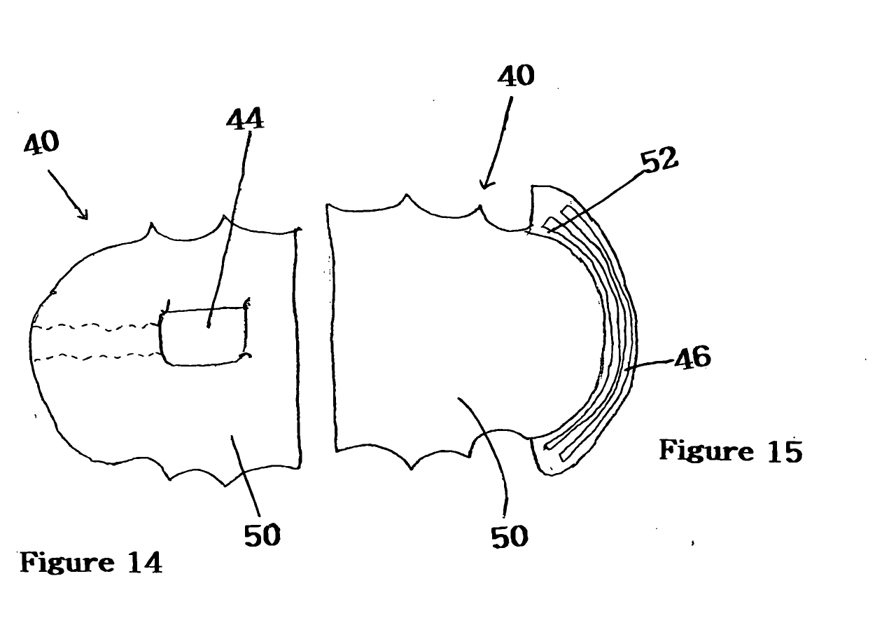

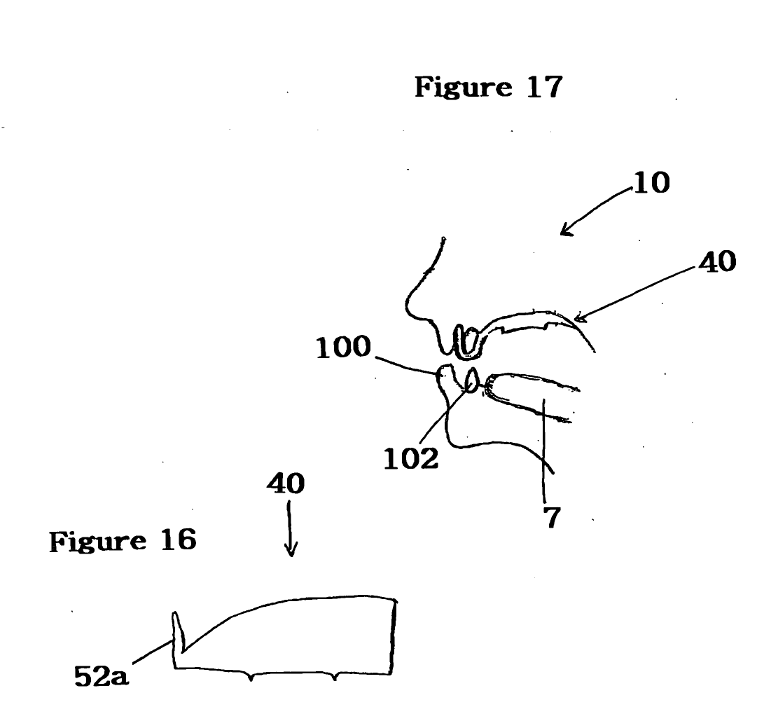

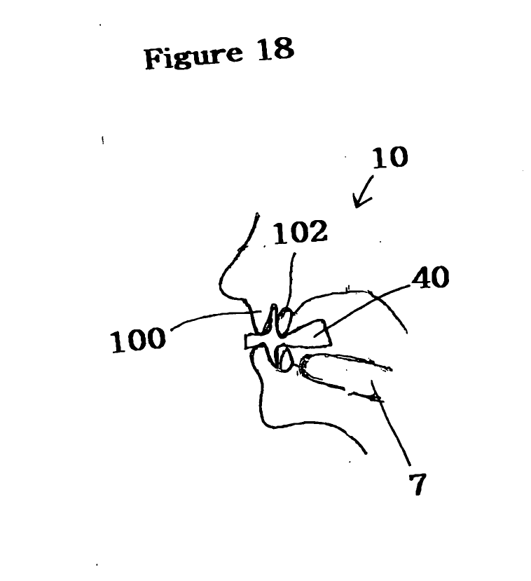

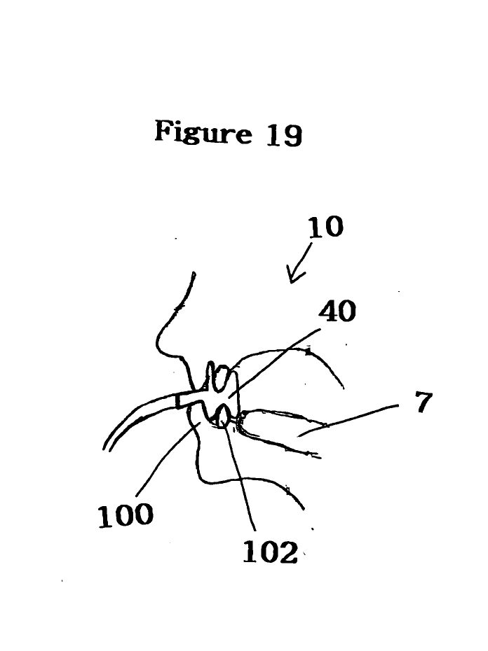

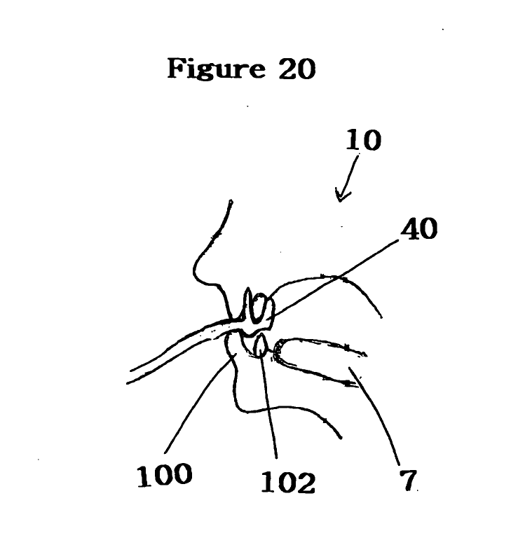

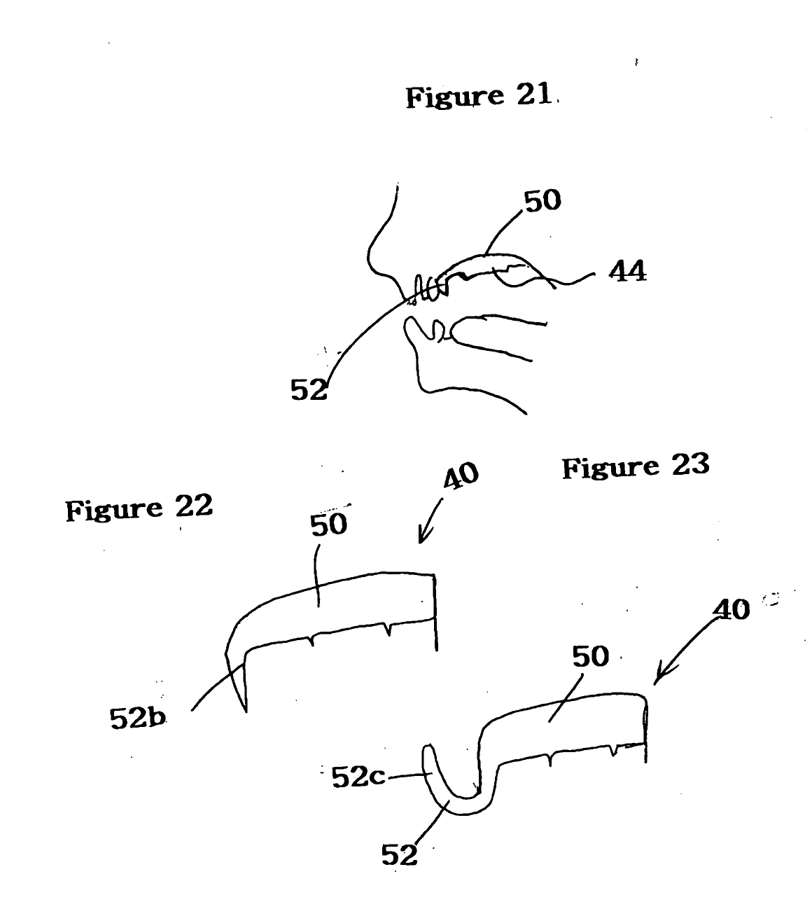

In another embodiment of the invention, as shown in FIGS. 3-6, light device 140 comprising a cold cathode fluorescent bulb or other bulb such as cold fusion bulb is placed directly into the patient's 10 mouth. It may be placed on top of the tongue 174, under the tongue 174 or between the tongue 100 and gums. Waterproof shell 170 can be oriented in substantially parallel alignment with the back of the tongue 174 or can be aligned substantially perpendicular to the back 174a of tongue 174 or it can be placed between the tongue 100 and the gums. Shell 170 is placed to irradiate the gums. Preferably, the shell 170 is made of a material which allows emission of UV light. Optionally, the shell 170 has a window the permits the emission of the UV radiation. If the shell 170 has a window, it is preferable that the window be oriented toward the bottom of the mouth. One or more low voltage cables 172 used to power the cold cathode fluorescent bulb. The low voltage cables are preferably attached to the shell at a waterproof connection 176. The bulb may have a shaft with one or more pins adapted to mate with the low voltage cable with a female connection end. Other known electrical connections can be utilized. It is preferable that the light source be removable for cleaning, sterilization and/or replacement of the bulb. In another alternative embodiment, the bulb and low voltage cable are an integral unit. In yet another alternative embodiment, the bulb and power supply are an integral unit. The light is preferably UV-A, UV-C or a combination thereof.

The light source may have a loop shape. The light source also may have has an angle to make it more comfortable for the patient.

Optionally, shell or bulb 170 is made of a flexible material. This will be more comfortable for the patient. Optionally, the flexible material allows the shell or bulb to mold to the patient's mouth.

In one embodiment, mouthguard 40 has channel 42 on either an upper 70 or lower side 72 or channels 42 on both the upper 70 and lower sides 72 of mouthguard 40. The gums or gums and teeth 102, if the patient still has teeth 102, are placed in channel 42 when the patient inserts mouthguard 40 into his or her mouth. One of more light sources 46 are housed in the channel 42. Mouthguard 40 and channels 42 may be of a U-shaped member 100 that fits over the majority of the population's teeth 102. Alternatively, mouthguard 40 and channels 42 may be molded specifically for the patient 10. A cast of the patient's mouth is made and mouthguard 40 is made from the cast. A specifically molded mouthguard 40 will be more comfortable for patient 10. Mouthguard 40 has a power supply 44 such as a battery pack. Power supply 44 powers light source 46. Alternatively, mouthguard 40 has a power supply connection 78 for connection to a power supply. The low voltage cables are preferably attached at a waterproof connection 176. The power supply connection may have a shaft with one or more pins adapted to mate with the low voltage cable with a female connection end. One or more low voltage cables 172 may be used to power the light source. Other known electrical connections can be utilized. Optionally, mouthguard 40 is connectable to a controller such as a computer.

Light source 46 may be a medical grade bulb, a cold cathode bulb, Preferably, the mouthguard 40 has an encapsulated light source 46. The encapsulated portion 80 is preferably made of a material that allows transmission of therapeutic light or has a window of material that allows for the transmission of UV light. It is preferable that the window be oriented toward the gums. One or more low voltage cables 172 may be used to power the cold cathode fluorescent bulb. Light source 46 may be situated on a front wall of the channel, a back wall of the channel, the bottom of the channel or a combination of those locations. It is preferable that the light source be removable for cleaning, sterilization and/or replacement of the bulb. In another alternative embodiment, the bulb and low voltage cable are an integral unit. In yet another alternative embodiment, the bulb and power supply are an integral unit. The light is preferably UV-A, UV-C or a combination thereof.

If mouthguard 40 has a power supply connection 78, it is preferable that the connection be at the patient's 10 mouth opening when the mouthguard is in the treatment position. A therapeutic wavelength or wavelengths of lights irradiates the gums. In addition, because capillaries in the gums are close to the surface, the blood is also treated with the therapeutic irradiation and the treated blood circulates to the heart and body. As the gums are irradiated, the progression of the gum disease may be slowed, the gums maybe maintained or the gums may improve. In addition, fatty plaque build-up in the circulatory system may be slowed or maintained. Because the 5.6 L of blood in a human body circulates through the body about 3 times every minute, large amounts of blood can be treated with photoluminescence by irradiating the gums.

Patient 10 can insert mouthguard 40 when a treatment is needed. Mouthguard 40 can be turned on and off manually. Alternatively, mouthguard could automatically turn off the light source 46 after a set treatment time, such as 20 minutes. In this way, patient 10 could put the mouthguard in while he slept or engaged in another activity and not have to monitor the treatment time. Mouthguard 40 could have a controller, computer or other smart interface that limits the number of treatments given time period, limits the total amount of treatment time in a given time period, automatically provides treatments, pulses the LEDs, or provides only particular wavelengths if the light pack has LEDs of various wavelengths. The computer or other smart interface could keep a treatment record. The computer or other smart interface could communicate wirelessly, via the Internet or through other electronic means to automatically update the doctor's treatment records. The computer could automatically adjust the treatment time based on input from the patient, the doctor, treatment records, or other data.

In another embodiment, mouthguard 40 comprises a plate 50 that fits the patient's 10 pallet with a flange 52 having one or more light sources 46. Plate 50 may be of a generic shape that fits the majority of the population's pallets or may be specifically molded to fit the patient's pallet. Flange 52 can be an upward flange 52a which fits over the patient's 10 teeth to irradiate the front of the gums or can be a downward flange 52b which irradiates the gums on the back of the teeth or groove flange 52c. Light source 46 is located on flange 52 such that it is directed toward the gums when the mouthguard is in the treatment position.

Light source may be a medical grade bulb, a cold cathode bulb, Preferably, light source 46 is encapsulated in flange 52. Encapsulated portion is preferably made of a material, which allows transmission of UV light or has a window of material that allows for the transmission of UV light. Mouthguard 40 has a power supply 44 such as a battery pack. Battery pack is preferably molded into plate. Power supply 44 powers light source 46. Alternatively, mouthguard 40 is connectable to a power supply 176. Optionally, mouthguard 40 is connectable to a controller such as a computer.

A therapeutic wavelength or wavelengths of light irradiates the gums. In addition, patient 10 can insert mouthguard when a treatment is needed. Mouthguard can be turned on and off manually. Alternatively, mouthguard could automatically turn off the light source after a set treatment time, such as 20 minutes. Mouthguard 40 could have a controller, computer or other smart interface that limits the number of treatments given time period, limits the total amount of treatment time in a given time period, automatically provides treatments, pulses the LEDs, or provides only particular wavelengths if the light pack has LEDs of various wavelengths. The computer or other smart interface could keep a treatment record. The computer or other smart interface could communicate wirelessly, via the Internet or through other electronic means to automatically update the doctor's treatment records. The computer could automatically adjust the treatment time based on input from the patient, the doctor, treatment records, or other data.

In another embodiment, mouthguard 40 is placed between the patient's 10 tongue 100 and gums 102. A light source is located on an inner surface of the mouthguard. Mouthguard 40 comprises a plug 60. Optionally, mouthguard 40 has a projection 62 on a second side 66 of plug 60. Projection 64 is used to help maintain mouthguard 40 in the patient's mouth. Projection 62 may be shaped to make it more comfortable for the patient. Plug 60 has a first side 64 having a power supply 44 or a power supply connector 78. Powers supply 44 or power supply connector is substantially centered on the first side 64 of plug 60. Plug 60 has a second side 66 having one or more light sources 46. Plug 60 is placed between the patient's tongue 100 and gums with the first side 64 facing the tongue 100 and the second side 66 facing the gums 102. Power supply or power supply connection preferably is located so that when the mouthguard 40 is in a treatment position power supply is exposed between patient's lips. Optionally, mouthguard 40 is connectable to a controller such as a computer.

Light source may be a medical grade bulb, a cold cathode bulb, Preferably, the mouthguard 40 encapsulates light source, the encapsulated portion is preferably made of a material which allows emission of UV light or has a window of material that allows for the transmission of UV light. One or more low voltage cables 172 used to power the cold cathode fluorescent bulb. The low voltage cables are preferably attached to the plug 60 at a waterproof connection 176. The power supply connection may have a shaft with one or more pins adapted to mate with the low voltage cable with a female connection end. Other known electrical connections can be utilized. It is preferable that the light source be removable for cleaning, sterilization and/or replacement of the bulb. In another alternative embodiment, the bulb and low voltage cable are an integral unit. In yet another alternative embodiment, the bulb and power supply are an integral unit. The light is preferably UV-A, UV-C or a combination thereof.

A therapeutic wavelength or wavelengths of lights irradiates the gums. In addition, because capillaries in the gums are close to the surface, the blood is also treated with the therapeutic irradiation and the treated blood circulates to the heart and body.

Patient 10 can insert mouthguard when a treatment is needed. Mouthguard can be turned on and off manually. Alternatively, mouthguard could automatically turn off the light source after a set treatment time, such as 20 minutes. Mouthguard 40 could have a controller, computer or other smart interface that limits the number of treatments given time period, limits the total amount of treatment time in a given time period, automatically provides treatments, pulses the LEDs, or provides only particular wavelengths if the light pack has LEDs of various wavelengths. The computer or other smart interface could keep a treatment record. The computer or other smart interface could communicate wirelessly, via the Internet or through other electronic means to automatically update the doctor's treatment records. The computer could automatically adjust the treatment time based on input from the patient, the doctor, treatment records, or other data.

I claim:

1. A gum illuminator comprising:
a mouthguard;
a power supply;
a light source powered by said power supply;
said light source adapted to emit radiation at one or more therapeutic wavelengths and said light source adapted to be placed under a patient's tongue 174.

2. The gum illuminator of claim 1 wherein the light source emits UV radiation.

3. The gum illuminator of claim 2 wherein the UV radiation is UV-A radiation, UV-C radiation or a combination thereof.

4. The gum illuminator of claim 1 wherein the light source is selected from the group consisting of cold cathode fluorescent bulb, cold fusion light source, fiber optic bundles, and LED bulbs.

5. The gum illuminator of claim 1 wherein the light source is a medical grade UV light bulb.

6. The gum illuminator of claim 3 wherein the mouthguard comprises a plate having a flange, said light source on said flange.

7. The gum illuminator of claim 15 wherein the power supply is integral with the plate.

8. The gum illuminator of claim 7 wherein the power supply is at least one battery.

9. The gum illuminator of claim 3 wherein the mouthguard comprises a substantially U-shaped member having at first substantially U-shaped channel on a first side, said light source is in said channel.

10. The gum illuminator of claim 9 further comprising a second substantially U-shaped channel of a second side of said mouthguard.

11. The gum illuminator of claim 9 wherein the channel comprises an inner wall, an outer wall and a floor, said walls are substantially parallel and said walls, and the light source is on the inner wall and directed toward the interior of the channel.

12. The gum illuminator of claim 9 wherein the channel comprises an inner wall, an outer wall and a floor, said walls are substantially parallel and said walls, and the light source is on the outer wall and directed toward the interior of the channel.

13. The gum illuminator of claim 9 wherein the channel comprises an inner wall, an outer wall and a floor, said walls are substantially parallel, and the light source is on the floor and directed toward the interior of the channel.

14. The gum illuminator of claim 9 wherein the channel comprises an inner wall, an outer wall and a floor, said walls are substantially parallel, and the light source is on the inner wall and the outer wall and is directed toward the interior of the channel.

15. The gum illuminator of claim 9 wherein the channel comprises an inner wall, an outer wall and a floor, said walls are substantially parallel, and the light source is on at least one wall and the floor and is directed toward the interior of the channel.

16. The gum illuminator of claim 10 wherein the channels comprises an inner wall, an outer wall and a floor, said walls are substantially parallel and said walls, and the light source is at least one wall of each channel and is directed toward the interior of the channel.

17. The gum illuminator of claim 9 wherein the power supply is on an outer curve of the U-shaped member.

18. The gum illuminator of claim 17 wherein the power supply is a battery.

19. The gum illuminator of claim 17 further comprising a power supply connector on an outer curve of the U-shaped member, said power supply connector connected to the power supply.

20. The gum illuminator of claim 10 wherein the power supply is on an outer curve of the U-shaped member.

21. The gum illuminator of claim 20 wherein the power supply is a battery.

22. The gum illuminator of claim 10 further comprising a power supply connector on an outer curve of the U-shaped member, said power supply connector connected to the power supply.

23. The gum illuminator of claim 3 wherein the mouthguard comprises a plug and the light source is on a first side of the plug.

24. The gum illuminator of claim 23 further comprising a nipple on the first side of the plug.

25. The gum illuminator of claim 23 wherein the power supply is on a second side of the plug.

26. The gum illuminator of claim 25 wherein the power supply is located at the center of the plug.

27. The gum illuminator of claim 25 wherein the power supply is a battery.

28. The gum illuminator of claim 23 further comprising a power supply connector on a second side of the plug, said power supply connector connected to the power supply.

29. The blood illuminator of claim 1 further comprising a controller.

30. The blood illuminator of claim 29 wherein the controller automatically controls the light source by pulsing the light, by automatically shutting off the light after a specified period of time, by automatically activating the light source at a specified time or by combinations thereof.

31. A method of illuminating blood comprising:
inserting the gum illuminator comprising a mouthguard and a light source into a patient's mouth,
directing radiation of one or more therapeutic wavelength to the patient's gums,
illuminating at least a portion of the patient's gums for a period of time with light.

32. The method of claim 31 further wherein the therapeutic wavelength is UV-A, UV-C or UV-A and UV-C light.

33. The method of claim 31 further comprising the step of placing at least a portion of the mouthguard between the patient's lips and gums.

34. The method of claim 31 further comprising the step of placing at least a portion of the mouthguard over the patient's gums.
